(19) United States
(12) Patent Application Publication
(10) Pub. No.: US 2025/0170425 A1
(43) Pub. Date: May 29, 2025

(54) RADIATION THERAPY SYSTEMS AND SHIMMING METHODS THEREOF

(71) Applicant: SHANGHAI UNITED IMAGING HEALTHCARE CO., LTD., Shanghai (CN)

(72) Inventors: Peng WANG, Shanghai (CN); Jiwen YANG, Shanghai (CN); Cheng NI, Shanghai (CN); Xing’en YU, Shanghai (CN)

(73) Assignee: SHANGHAI UNITED IMAGING HEALTHCARE CO., LTD., Shanghai (CN)

(21) Appl. No.: 18/959,527

(22) Filed: Nov. 25, 2024

(30) Foreign Application Priority Data
Nov. 23, 2023 (CN) 202323173465.0

(51) Int. Cl. A61N 5/10; G01R 33/3875; G01R 33/56

(52) U.S. Cl. CPC A61N 5/1081; A61N 5/1049; G01R 33/3875; G01R 33/5608; A61N 2005/1055; A61N 2005/1094

(57) ABSTRACT

Disclosed is a radiation therapy system and shim method thereof. The system includes: a magnet container, a therapy assembly including one or more magnetic components; and a plurality of target shim members. Each of the plurality of target shim members has parameter characteristics and spatial characteristics, and at least one of the parameter characteristics and the spatial characteristics of at least two of the plurality of target shim members is different. The method includes: obtaining a first main magnetic field; and for each of a plurality of first magnetic field sub-regions, in response to a difference between a magnetic field strength of the first magnetic field sub-region and a magnetic field strength of another first magnetic field sub-region adjacent to the first magnetic field sub-region being greater than a first preset threshold, determining a plurality of target shim members in the radiation therapy system.

### CROSS-REFERENCE TO RELATED APPLICATIONS

This application claims priority to Chinese application No. 202323173465.0 filed on Nov. 23, 2023, the entire contents of which are incorporated herein by reference.

### TECHNICAL FIELD

The present disclosure relates to the field of medical technology, and in particular relates to a radiation therapy system and a shimming method thereof.

### BACKGROUND

A radiation therapy device performs a radiation therapy on a target (e.g., a tumor) by emitting radiation beam(s). A magnetic resonance imaging (MRI) device is capable of performing various types of fluoroscopic examinations on patients through imaging.

If the radiation therapy device and the MRI device are integrated in a same system, due to a rotatable magnetic component disposed in the radiation therapy device, on the one hand, the magnetic component may cause a disturbance to a magnetic field of an imaging region; on the other hand, a spatial position of the magnetic component may change relative to the magnetic field of the imaging region when rotating, thus causing a disturbance to the magnetic field of the imaging region, resulting in a deterioration of a magnetic field evenness of the imaging region, and in turn affects an imaging effect.

Accordingly, there is a need to provide a radiation therapy system and a shimming method thereof.

### SUMMARY

One or more embodiments of the present disclosure provide a radiation therapy system including: a magnet container configured to form an imaging region; a therapy assembly including one or more magnetic components, the one or more magnetic components inducing an interfering magnetic field; and a plurality of target shim members. The target shim members are disposed on the radiation therapy system, each of the plurality of target shim members has parameter characteristics and spatial characteristics, and at least one of the parameter characteristics and the spatial characteristics of at least two of the plurality of target shim members is different; and the plurality of target shim members are configured to generate a compensating magnetic field, the compensating magnetic field being configured to at least partially counteract the interfering magnetic field.

One or more embodiments of the present disclosure provide a shimming method of a radiation therapy system, including: obtaining a first main magnetic field within an imaging region, the first main magnetic field including a plurality of first magnetic field sub-regions; and for each of the plurality of first magnetic field sub-regions, in response to a difference between a magnetic field strength of the first magnetic field sub-region and a magnetic field strength of another first magnetic field sub-region adjacent to the first magnetic field sub-region being greater than a first preset threshold, determining one or more target shim members in the radiation therapy system.

### BRIEF DESCRIPTION OF THE DRAWINGS

The present disclosure is further illustrated in terms of exemplary embodiments. These exemplary embodiments are described in detail with reference to the drawings. These embodiments are not limiting, and in these embodiments, the same numbering denotes the same structure, wherein

FIG. 1 is a schematic diagram illustrating a portion of a three-dimensional (3D) structure of an exemplary radiation therapy system according to some embodiments of the present disclosure;

FIG. 2 is a local cross-sectional view of an exemplary radiation therapy system according to some embodiments of the present disclosure;

FIG. 3 is a schematic diagram illustrating a three dimensional (3D) structure of an exemplary radiation therapy system according to some other embodiments of the present disclosure;

FIG. 4 is a schematic diagram illustrating an exemplary fixing member in a radiation therapy system according to some embodiments of the present disclosure;

FIG. 5 is a schematic diagram illustrating a structure of an exemplary fixing member according to some embodiments of the present disclosure;

FIG. 6 is a schematic diagram illustrating a structure of an exemplary fixing member according to some other embodiments of the present disclosure;

FIG. 7 is a schematic diagram illustrating an exemplary A-A sectional view of the fixing member shown in FIG. 6 according to other embodiments of the present disclosure;

FIG. 8 is a schematic diagram illustrating a 3D structure of an exemplary radiation therapy system according to other embodiments of the present disclosure; and

FIG. 9 is a schematic diagram illustrating an application manner of a parameter model according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for those skilled in the art to apply the present disclosure to other similar scenarios in accordance with these drawings without creative labor. Unless obviously obtained from the context or the context illustrates otherwise, the same numeral in the drawings refers to the same structure or operation.

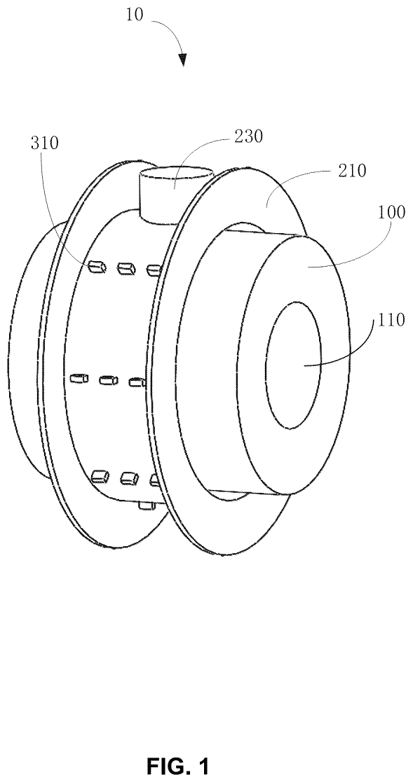

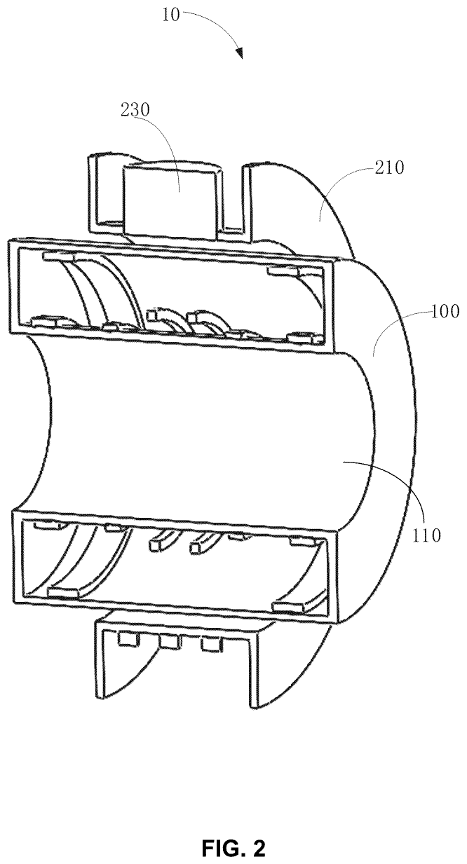

FIG. 1 is a schematic diagram illustrating a portion of a three-dimensional (3D) structure of an exemplary radiation therapy system according to some embodiments of the present disclosure. FIG. 2 is a local cross-sectional view of an exemplary radiation therapy system according to some embodiments of the present disclosure.

In some embodiments, as shown in FIGS. 1 and 2, a radiation therapy system 10 includes a magnet container 100, a therapy assembly, and a plurality of target shim members.

The magnet container 100 forms an imaging region 110. In some embodiments, a superconducting magnet (not shown) is disposed inside the imaging region 110, and the superconducting magnet forms a main magnetic field. The superconducting magnet is capable of generating a relatively high intensity magnetic field and has a strong magnetic field stability. The superconducting magnet is used in, for example, the radiation therapy system where a high precision is required. The superconducting magnet is an example of a main magnetic coil. In some embodiments, the superconducting magnet may include a plurality of main magnetic coils, and the plurality of main magnetic coils may be arranged around a central axis of the gantry 210. The main magnetic coil is used to generate a uniform main magnetic field in a specific field in and/or outside the accommodation cavity. The plurality of main magnetic coils are connected to each other through wires. When a current passes through the main magnetic coil(s), the magnetic field is generated in the imaging region 110. It should be noted that a coverage area of the main magnetic field is not limited to the imaging region 110.

As the superconducting magnet needs to be maintained at an extremely low temperature during operation to remain superconductive, the superconducting performance of the superconducting magnet is usually achieved by using extremely low temperature coolant such as liquid helium or liquid nitrogen. In some embodiments, the radiation therapy system 10 further includes a cold head (not shown). The cold head is disposed at an end of the superconducting magnet. The cold head typically includes an insulating material and a cooling system. The cold head is configured to deliver the coolant into the superconducting magnet and maintain a stable operation temperature of the superconducting magnet.

For some other application scenarios in which a therapy with a lower magnetic field strength or a miniaturized device is required, a permanent magnet or an electromagnet may be used as an alternative to the superconducting magnet.

In some embodiments, a plurality of magnetic shielding coils (not shown) are disposed within the imaging region 110. The plurality of magnetic shielding coils are disposed around the central axis of the gantry 210. Taking the superconducting magnet as an example of the main magnetic coil, a current direction of the magnetic shielding coil is opposite to the current direction of the main magnetic coil. By setting an inner diameter of the magnetic shielding coil greater than an outer diameter of the main magnetic coil, the magnetic field generated by the main magnetic coil outside an imaging region (also referred to as a detection region) is reduced or eliminated. The main magnetic coil and the magnetic shielding coil may be separated or interconnected. The main magnetic coil and the magnetically shielded coil may be annular coils disposed along the central axis of the gantry 210.

In some embodiments, one or more gradient coils (not shown) are disposed in the imaging region 110. The gradient coil(s) are arranged around the central axis of the gantry 210. The gradient coil(s) are used to generate a gradient field, which is superimposed on the main magnetic field to distort it, such that a magnetic orientation of protons of an object in the imaging region 110 varies as a function of their positions within the gradient field, thereby encoding spatial information into echo signals generated from a detected portion of the object.

The therapy assembly is a main component used for radiation therapy.

In some embodiments, the therapy assembly includes a gantry 210, which is a support structure for supporting, and the gantry 210 is rotatable. In some embodiments, the gantry 210 is disposed around the imaging region 110. In some embodiments, the gantry 210 is disposed on an outer side/inner side of the magnet container 100.

In some embodiments, the therapy assembly includes a therapy head 230, and a linear accelerator is disposed inside the therapy head 230. The linear accelerator is configured to accelerate electrons to generate a radiation beam. In some embodiments, the linear accelerator is mounted on the gantry 210. The therapy head 230 is used to perform a radiation therapy (e.g., by emitting radiation beam(s)) on a patient. In some embodiments, the therapy head 230 includes the linear accelerator. Exemplarily, the therapy head 230 includes a radiation source that emits the radiation beam(s). An exemplary radiation beam includes a particle ray beam, a photon ray beam, etc. Exemplary particle rays include neutrons, protons, electrons, muons, heavy ions, alpha rays, etc., or any combination thereof. Exemplary photon rays include X-rays, gamma rays, ultraviolet rays, lasers, etc., or any combination thereof. A specific structure and a dispose position of the therapy assembly is set according to actual needs, so as to emit the radiation beam(s) at a specific angle for the radiation therapy.

In some embodiments, the therapy assembly is operably connected to an magnetic resonance imaging (MRI) device. The MRI device is configured to acquire image data of an object prior to, during, or after at least a portion of the radiation therapy performed on the object. The therapy assembly (also referred to as a radiation therapy device) delivers the radiation beam(s) to a target region of the object based on the image data of the object provided by the MRI device. The object is placed on a therapy table, and the therapy table supports the object for imaging performed by the MRI device and/or for the radiation therapy performed by the radiation therapy device. The therapy table moves along an axial direction of the gantry. If the object needs to be treated, the therapy table carrying the object is moved to a therapy position of the radiation therapy device. If the object needs to be imaged, the therapy table carrying the object is moved to an imaging position of the MRI device.

A magnetic component is a structure with magnetism. The magnetic field generated by the magnetic component(s) interfere with the magnetic container.

In some embodiments, the therapy assembly includes the magnetic component(s). For example, structures like the linear accelerator, a power supply, etc. have a structure containing ferromagnetic materials, i.e., the magnetic component(s).

In some embodiments, the therapy assembly includes the gantry 210. In some embodiments, the plurality of target shim members are unevenly distributed on the gantry 210.

In some embodiments, the magnetic component(s) are disposed as a portion of the gantry 210. Component(s) (e.g., a motor, a bearing, etc.) mounted on the gantry 210 includes ferromagnetic substances, i.e., the magnetic component(s). In some embodiments, the magnetic component(s) generate an interfering magnetic field, which generates interferences. The interfering magnetic field includes the magnetic field generated by the magnetic component(s) of the radiation therapy system 10 (e.g., the magnetic component(s) within the therapy assembly), and the magnetic field generated when there is a relative position change of the magnetic component(s). The interfering magnetic field interferes with the main magnetic field.

In some embodiments, the plurality of target shim members are disposed on the radiation therapy system. In some embodiments, each of the plurality of target shim members has parameter characteristics and spatial characteristics, and at least one of the parameter characteristics and the spatial characteristics of at least two of the plurality of target shim members is different. The parameter characteristics at least include a geometric characteristic of each of the plurality of target shim members, the geometric characteristic refers to a physical state of the target shim member, that is, the target shim members have same or different outlines, volumes, etc. The spatial characteristics at least include position information. The position information includes a distance between each adjacent two target shim members of the plurality of target shim members.

The one or more target shim members are configured to generate a compensating magnetic field. The compensating field is used to at least partially counteract the interfering magnetic field. The compensating magnetic field, i.e., the magnetic field that compensates for the interfering magnetic field, reduces the interference of the interfering magnetic field generated by the magnetic component(s) of the therapy assembly on the magnet container 100.

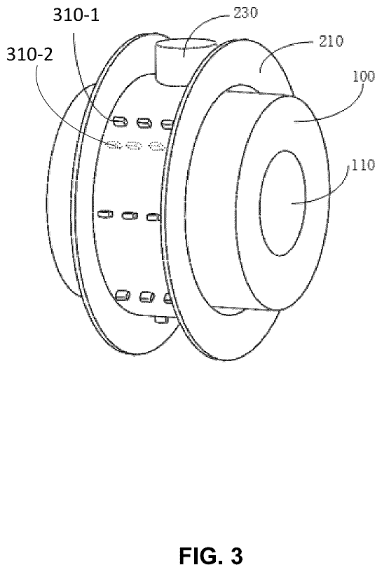

In some embodiments, the interfering magnetic field generated by the magnetic component(s) are uneven. For example, a position where the magnetic component(s) are densely distributed has a higher magnetic field strength, and the closer the position is to the magnetic component(s), the higher the magnetic field strength. Therefore, to maintain an evenness of the magnetic field, it is necessary to correspondingly dispose target shim members. For example, the position where the magnetic component(s) are densely distributed are disposed with densely distributed target shim members (see FIG. 3), and the position closer to the magnetic component(s) are disposed with target shim members with greater geometric shapes. For another example, the target shim members are distributed with equal distances, and the closer the target shim members are to the magnetic component(s), the greater volume of the target shim members; or, not only are the target shim members closer to the magnetic component(s) more densely distributed, but area of the target shim members closer to the magnetic component(s) is also larger than the area of the target shim members away from the magnetic component(s).

In some embodiments, the one or more target shim members include one or more first shim members 310 and/or one or more him members 400.

In some embodiments, the one or more target shim members are capable of rotating synchronously with the gantry 210. The rotation of the magnetic component(s) of the therapy assembly induces a spatial position change of the magnetic component(s) with respect to the magnetic field of the magnet container 100, which has an effect on the magnetic property. The one or more target shim members rotate at the same time as the magnetic component(s) of the therapy assembly, so that the effect of the magnetic component(s) of the therapy assembly on the magnetic property and the effect of the target shim member(s) on the magnetic property cancel each other out.

In some embodiments of the present disclosure, the effect of the magnetic component(s) in the therapy assembly on the main magnetic field is reduced by disposing one or more target shim members. The compensating magnetic field generated by the synchronized rotation of the one or more target shim members with the gantry at least partially cancels out the interfering magnetic field.

In some embodiments, the radiation therapy system 10 includes a shim gantry. The shim gantry provides a supporting structure for disposing the target shim member(s), i.e., the target shim member(s) are mounted on or integrated into the shim gantry. In some embodiments, the shim gantry is connected to the gantry 210, and the shim gantry rotates synchronously with the gantry 210.

The disposing of the shim gantry allows for easy disassembly and a routine maintenance of the target shim member(s), while providing more optional positions for placing the target shim member(s).

In some embodiments, the one or more target shim members include at least one of a permanent magnet, a ferromagnetic element, and an electromagnetic coil. The permanent magnet safeguards the magnetic property of the target shim member(s) from being eliminated over time. The ferromagnetic element makes it easy to adjust a strength of the magnetic property of the target shim member(s). The electromagnetic coil shows no magnetic property when not in use, so as to avoid a demagnetization of the magnetic component(s) in the therapy assembly. Options can be made to use different types of target shim members with different effects according to the actual needs, and the use of the target shim member(s) can be expanded.

In some embodiments, the one or more target shim members include one or more first shim members 310. The one or more first shim members 310 are distributed on the gantry 210.

In some embodiments, the one or more first shim members 310 are configured to counteract the interfering magnetic field at a first target position. The first target position is located within the imaging region 110, and the first target position is a position within the imaging region 110 to be imaged. In some embodiments, the first target position is located in the imaging region 110. At the first target position, directions of a first compensating magnetic field and the interfering magnetic field are different.

FIG. 3 is a schematic diagram illustrating a three dimensional (3D) structure of an exemplary radiation therapy system according to some other embodiments of the present disclosure.

In some embodiments, as shown in FIG. 3, a first shim members 310-1 is distributed on an outer side of the gantry 210. In some embodiments, as shown in FIG. 3, a first shim members 310-2 is distributed on an inner side of the gantry 210.

In some embodiments, the one or more first shim members 310 are distributed in a circumferential array around the gantry 210.

In some embodiments, a distribution density formed by a distribution of the one or more first shim members 310 disposed close to the magnetic components is greater than a distribution density formed by a distribution of the one or more first shim members 310 disposed away from the magnetic components.

By disposing more densely distributed first shim pieces close to the position

where the magnetic component(s) are located, an evenness of the magnetic field is maintained and a shim effect is improved.

In some embodiments, a plurality of target shim members include one or more shim members. The one or more shim members are shim members disposed in a peripheral region of a therapy assembly. In some embodiments, the one or more second shim members 400 are distributed on the therapy assembly. The one or more second shim members are configured to counteract the interfering magnetic field at a second target position. The second target position is located on the therapy assembly or in the region around the therapy assembly.

In some embodiments, the one or more first shim members 310 and/or the one or more second shim members 400 are unevenly distributed along the gantry 210. It is appreciated that the magnetic components in the therapy assembly are generally non-uniformly distributed along the gantry 210 and that the interfering magnetic field generated by the magnetic components diminishes as a distance increases. For example, if one of the motors on the gantry 210 is magnetic (i.e., the motor is a magnetic component), then for the gantry 210 as a whole, an effect of the interfering magnetic field is relatively high on a side close to the motor, and the effect of the magnetic field is relatively low on a side away from the motor. Thus a nonuniformity of the interfering magnetic field is generated. By unevenly distributing the target shim members along the gantry 210, i.e., laying at least one type of the one or more first shim members 310 and the one or more second shim members 400 at one or more specific positions on the gantry according to the nonuniform distribution of the interfering magnetic field generated by the magnetic component(s), the nonuniformly distributed interfering magnetic field may be counteracted.

In some embodiments, as shown in FIGS. 1 and 2, a plurality of first shim field members 310 are distributed in an array along a circumferential direction of the gantry 210. Along the circumferential direction, distances between two adjacent first shim field members 310 are equal, i.e., the plurality of first shim field members 310 are distributed evenly along the circumferential direction of the gantry 210. Understandably, the plurality of first shim field members 310 distributed in the array can generate a uniform magnetic field that counteracts the interfering magnetic field within the imaging region 110 to reduce the magnetic field nonuniformity. By disposing the plurality of first shim field members 310 along the circumferential direction of the gantry 210, the compensating magnetic field generated therefrom is made to be distributed more evenly along the circumferential direction, so as to counteract the interfering magnetic field to reduce the interference of the interfering magnetic field on the main magnetic field in the imaging region, so that the uniformity of the main magnetic field is improved.

It will be appreciated that, in other embodiments, along the circumferential direction of the gantry, the distances between two adjacent first shim members are unequal, i.e., the plurality of first shim members are disposed unevenly in the circumferential direction of the gantry. The specific arrangement is adopted based on requirements of actual uses, and an optimal arrangement of the plurality of first shim members is determined through a simulation of the interfering magnetic field using an optimization algorithm.

By disposing the plurality of first shim field members 310 along the circumferential direction on an outer circumferential wall of the gantry 210, the plurality of first shim field members 310 are made to generate the compensating magnetic field along the circumferential direction so as to counteract the interfering magnetic field, thereby reducing the effect of the therapy assembly on the main magnetic field in the imaging region. Exemplarily, the linear accelerator generates a magnetic field effect A on the imaging region, which is understood as a magnetic field distribution. For the optimized arrangement of the first shim members 310, reference may be made to Halbach Array, so as to generate a magnetic field effect-A, or close to-A, in the imaging region. In this way, the effect of the linear accelerator on the main magnetic field in the imaging region is counteracted or reduced. At the same time, as both the first shim members 310 and the linear accelerator rotate synchronously with the gantry 210, there is no change in the relative position between the first shim members 310 and the linear accelerator, i.e., the counteracting effect of the first shim member 310 on the interfering magnetic field of the linear accelerator always exists, thereby reducing the interference of the linear accelerator on the main magnetic field, and making the magnetic field uniformity in the imaging region better, and thus ensuring the imaging effect.

In some embodiments, a compensating effect of the first compensating magnetic field on the interfering magnetic field is confirmed by testing the magnetic field uniformity. For example, for a sphere with a center of the magnet container 100 as a center and with a preset diameter, a detector is disposed on a spherical surface to measure a magnetic field distribution B, and the magnetic field nonuniformity is evaluated by (Bₘₐₓ−Bₘᵢₙ)/Bₘᵢₙ*1e6 (in ppm), the greater the value of the magnetic field nonuniformity, the worse the magnetic field uniformity. Exemplarily, on the spherical surface with a diameter of 50 cm, a nonuniformity <±1 ppm is regarded as reaching the preset compensating effect.

In some embodiments, the magnetic field uniformity of the interfering magnetic field is measured before shimming, the corresponding first compensating magnetic field is designed according to the magnetic field nonuniformity, and the magnetic field nonuniformity is tested again after shimming, until an expected compensating effect is achieved, and the shimming is completed.

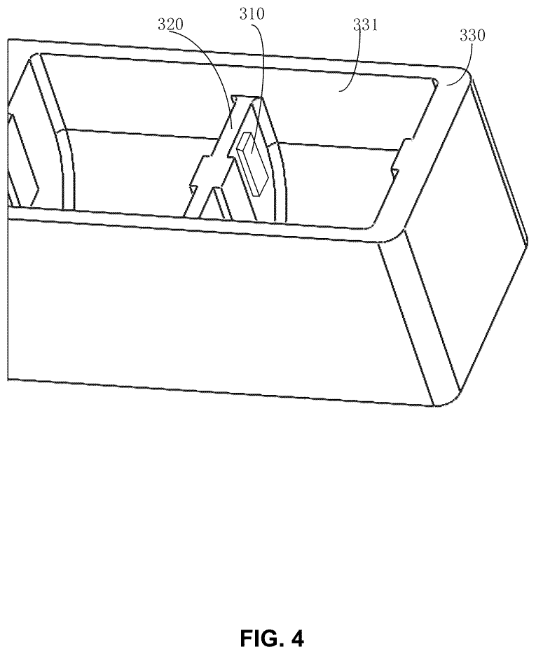

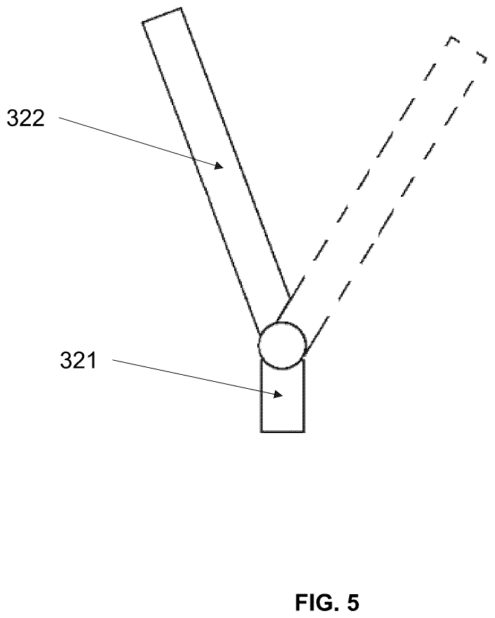

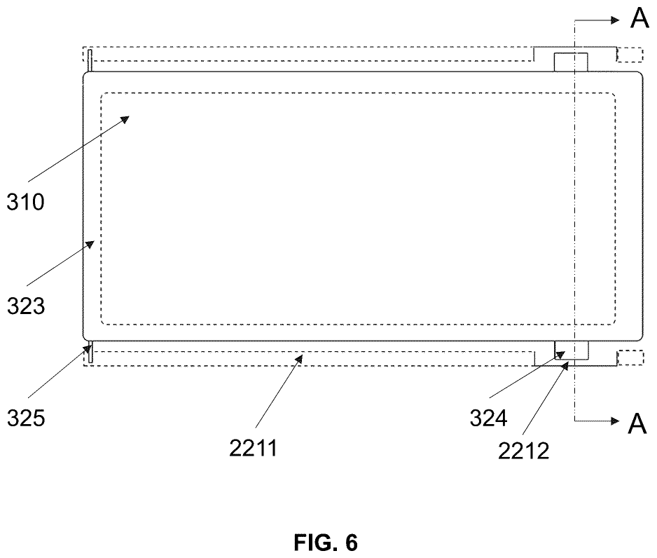

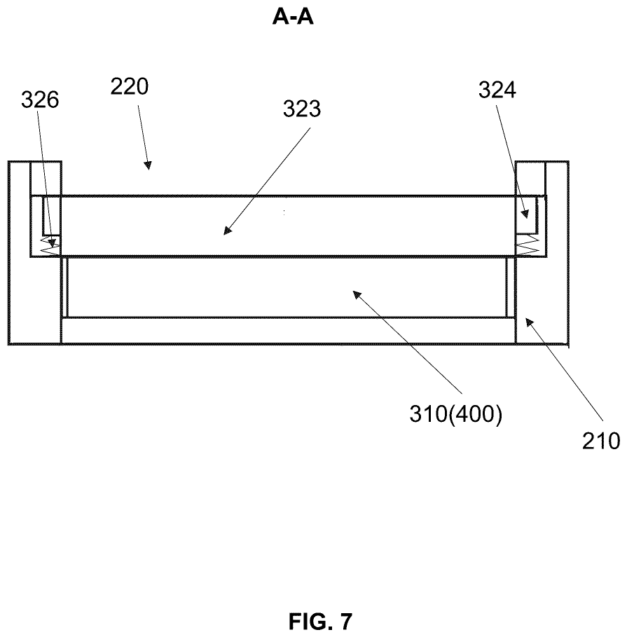

FIG. 4 is a schematic diagram illustrating an exemplary fixing member in a radiation therapy system according to some embodiments of the present disclosure. FIG. 5 is a schematic diagram illustrating a structure of a fixing member according to some embodiments of the present disclosure. FIG. 6 is a schematic diagram illustrating a structure of an exemplary fixing member according to some other embodiments of the present disclosure. FIG. 7 is a schematic diagram illustrating an exemplary A-A sectional view of the fixing member shown in FIG. 6 according to other embodiments of the present disclosure.

In some embodiments, as shown in FIGS. 1 and 4, the radiation therapy system 10 further includes one or more fixing members 320 configured to fix the one or more first shim members 310 and the one or more second shim members 400. In some embodiments, the one or more fixing members 320 are connected to a surface of the gantry 210 to fix the one or more first shim members 310. In some embodiments, the one or more fixing members 320 are connected to a surface of the therapy assembly to fix the one or more second shim members 400.

The one or more fixing members 320 are used to fix one or more target shim members to corresponding positions on the gantry 210. In some embodiments, a groove is disposed on the surface of the gantry 210 or the therapy assembly, and the one or more first shim members 310 or the one or more second shim members 400 are fixedly disposed in the groove through the one or more fixing members 320. The one or more fixing members 320 may have various structures. For example, the one or more fixing members 320 may include fixing plates to which the one or more first shim members 310 or the one or more second shim members 400 are bonded, and the fixing plates are embedded in the aforementioned groove. Cross-sectional shapes of the one or more first shim members 310 or the one or more second shim members 400 include regular shapes such as circles or squares, which facilitates the producing of the one or more first shim members 310 or the one or more second shim members 400. At the same time, a contact area of the one or more first shim members 310 or the one or more second shim members 400 and the fixing members 320 is increased, which improves a connection effect therebetween.

In some embodiments, as shown in FIG. 5, at least one of the one or more fixing members 320 includes a connection element 321 and a fixing support 322.

The connection element 321 is configured to connect the fixing support 322 to the gantry 210 or the therapy assembly. In some embodiments, at least one first connection element connects the gantry 210 and at least one first fixing support. In some embodiments, at least one second connection element connects the therapy assembly and at least one second fixing support.

The fixing support 322 is configured to fix the at least one of the one or more first shim members 310 or the one or more second shim members 400. For example, the fixation is implemented by bonding, threaded connection, etc. In some embodiments, the at least one first fixing support fixes the one or more first shim members 310. In some embodiments, the at least one first fixing support fixes the one or more second shim members 400.

In some embodiments, the fixing support 322 is a rod-like member with a certain length. The fixing support 322 has a variety of shapes, such as a flat elongated shape, a rounded rod shape, a shape with a variable section, etc.

In some embodiments, the fixing support 322 and the connection element 321 are fixedly connected, for example, integrated, etc.

In some embodiments, as shown in FIG. 5, the fixing support 322 rotates with respect to the connection element 321 such that an arbitrary angle is formed between the fixing support 322 and the connection element 321. At the same time, the relative position of the fixing support 322 and the connection element 321 are fixed, such that the angle between the fixed support 322 and the connection elements 321 is maintained at any angle.

In some embodiments, a direction in which the connection element 321 is set determines a rotation direction of the fixed support 322. For example, according to a setting position of the connection element 321 with respect to the gantry 210, the fixed support 322 rotates axially along the gantry 210, or the fixed support 322 rotates radially along the gantry 210 or at other angles. By using the fixed support 322 capable of rotating with respect to the connection element, it is easy to adjust the positions of the one or more first shim members, thereby further ensuring the uniformity of the magnetic field.

In some embodiments, the connection element 321 and the gantry 210 is made of a material including a damping material, e.g., a fluid damping material, a friction damping material, etc.

By using the connection element and the fixed support, a mounting region of the first shim member(s) are extended, which facilitates the disassembly, maintenance, and position adjustment of the first shim member(s), and the uniformity of the magnetic field is guaranteed.

In some embodiments, at least one of the one or more first shim members 310 includes a first shim coil. The gantry 210 is equipped with one or more fixing members 320, and each fixing member 320 is equipped with at least one first shim coil.

In some embodiments, when the gantry 210 is rotating, a current passes through the first shim coil. When the gantry 210 is at rest (i.e., is not rotating), no current passes through the first shim coil.

The first shim coil is an electromagnetic coil disposed on the gantry. When the gantry rotates, the current passes through the first shim coil, and at the same time, a compensating magnetic field is generated. When the gantry is at rest, no compensating magnetic field is generated. By controlling whether or not the current passes through the first shim coil, whether or not the first shim coil is energized is controlled, so as to avoid an adverse effect on the other magnetic components (e.g., causing degaussing of the other magnetic components) caused by a long existing of the magnetic field.

The connection between the connection element 321 and the gantry 210 is achieved through various ways. For example, the connection element 321 is a plug-in, and a groove is disposed in the gantry 210, and the connection is made by inserting the connection element 321 into the groove. As another example, the connection element 321 is snapped to the gantry 210. As a further example, the connection element 321 is connected with the gantry 210 through a threaded connection.

In some embodiments, as shown in FIGS. 6 and 7, the gantry 210 or the therapy assembly is provided with one or more fixing grooves 220. Structures of the fixing grooves 220 are adapted to the structures of the one or more fixing members 320. Each of the one or more fixing grooves 220 is capable of connecting the one or more first shim members 310 or the one or more second shim members 400 via the one or more fixing members 320.

The one or more fixing grooves 220 are configured to accommodate the one or more fixing members 320.

In some embodiments, as shown in FIG. 6, two limiting grooves 221 are disposed on two sides of a fixing groove 220, and each of the limiting grooves 221 includes a first groove section 2211 and a second groove section 2212.

The first groove section 2211 and the second groove section 2212 are configured to limit and guide a first limiting member 324 in at least one of the one or more fixing members 320. The first groove section 2211 is connected to the second groove section 2212, the first groove section 2211 is disposed within the gantry 210 or the therapy assembly and is not connected to an exterior, and the second groove section 2212 is disposed on the gantry 210 or the surface of the therapy assembly and connected to the exterior.

In some embodiments, as shown in FIGS. 6 and 7, at least one of the one or more fixing members 320 includes a limiting body 323 and at least two first limiting members 324. The limiting body 323 is disposed above the fixing groove 220 and the at least two first limiting members 324 are disposed on two sides of the limit body 323.

In some embodiments, the first limiting member 324 is capable of sliding relative to the limiting member 323 in an extension direction of the first groove section 2211 and the second groove section 2212. In some embodiments, a size of the fixing groove 220 matches a size of the limiting body 323, so that the limiting body 323 is embedded in the fixing groove 220. The limiting body 323 has a greater size than the one or more first shim members 310 or the one or more second shim members 400, so that at least one of the one or more first shim members 310 or at least one of the one or more second shim members 400 is pressed and limited by the limiting body 323 in the fixing groove 220.

In some embodiments, the limiting body 323 has a hollow structure, i.e., an edge position of at least one of the one or more first shim members 310 or at least one of the one or more second shim members 400 is limited by the limiting body 323, and an intermediate region is exposed to facilitate a propagation of the magnetic field, and to facilitate observing whether the first shim member 310 or the second shim member 400 is disposed thereon.

The one or more first limiting members 324 are configured to control opening and closing of the one or more fixing members 320. In some embodiments, at least one first limiting member 324 is disposed on each side of the two sides of the limiting body 323. In some embodiments, the counts of the first limiting members 324 on two sides are the same or different.

In some embodiments, the first limiting member 324 is capable of sliding relative to the limiting body 323 along an extension direction of the first groove section 2211 and the second groove section 2212.

In some embodiments, the fixing member 320 is capable of sliding between a first limiting position and a second limiting position. When the one or more fixing members 320 are located in the first limiting position, at least two of the first limiting members 324 are located in the first groove section 2211. At this time, the first limiting members 324 are limited by the first groove section 2211, thereby limiting the one or more fixing members 320 in the fixing groove 220, and the limiting body 323 presses the one or more first shim members 310 or the one or more second shim members 400 in the fixing groove 220. When the one or more fixing members 320 are located in the second limiting position, at least two of the first limiting members 324 are located in the second groove section 2212, and the first limiting members 324 are not limited by the second groove section 2212, and thus the limit body 323 is not limited by the limit groove 220, and the limit body 323 is unable to press the one or more first shim members 310 or the one or more second shim members 400 in the fixing groove 220.

In some embodiments, as shown in FIG. 6, the fixing member 320 further includes two second limiting members 325. The second limiting members 325 are symmetrically and fixedly disposed at ends of both sides of the limiting body 323, and the second limiting members 325 are disposed within the first groove section 2211 when the one or more fixing members 320 are in the first limiting position and the second limiting position.

The second limiting members 325 are components for preventing the one or more fixing members 220 from falling off the gantry 210 or the therapy assembly. In some embodiments, the second limiting members 325 have columnar structures, such that the first groove section 2211 limits a position of the second limiting members 325 to prevent the one or more fixing members 220 from falling off the gantry 210 or the therapy assembly.

In some embodiments, as shown in FIG. 7, the first limiting member 324 is equipped with a spring 326, which is compressed when the one or more fixing members 320 are in the first limiting position. When the fixing member 320 is in the second limiting position, the spring 326 imposes an elastic force on the fixed member 320 away from the gantry 210 or the therapy assembly. For example, the spring 326 is disposed on a side of the first limiting member 324 close to the gantry 210 or the therapy assembly, and when the first limiting member 324 is disposed in the second groove section 2212, by means of the spring 326, the one or more fixing members 320 on the corresponding side are automatically pushed in a direction departs from the gantry 210 or the therapy assembly to facilitate operation.

In some embodiments of the present disclosure, through using one or more fixing grooves, the mounting and disassembly of the one or more first shim members are facilitated. Through disposing the second limiting members on the one or more fixing members, the opening and closing of the one or more fixing members are facilitated. At the same time, when disassembling, the one or more fixing members do no fall due to a restriction of the second limiting members. By disposing the spring on the first limiting member, one side of the one or more fixing members automatically moves when the one or more fixing members are opened, which facilitates operation.

In some embodiments, at least one of the one or more first shim members 310 includes a first shim magnet, and the radiation therapy system further includes a shim shield member 340. The shim shielding member 340 is configured to shield a magnetic field generated by the first shim magnet when the gantry 210 is at rest, and un-shield the magnetic field generated by the first shim magnet when the gantry 201 is rotating.

The shim shielding member 340 refers to a component that isolates useless magnetic forces. The shim shielding member 340 may include a shielding plate, a magnetic shielding coil, etc.

In some embodiments, the shim shielding member 340 is the magnetic shielding coil disposed around the first shim magnet. When the first shim magnet is not in use, the shim field shielding member 340 shields the magnetic field of the first shim magnet to avoid the magnetic components of the therapy assembly from demagnetization.

In some embodiments of the present disclosure, as the first shim magnet and the therapy assembly generate opposite magnetic fields, which results in the magnetic properties of the magnetic components in the therapy assembly being weakened and affects a normal use, the shim field shielding member is configured to prevent the first shim magnet and the therapy assembly from being in a same space for a long period of time.

In some embodiments, the radiation therapy system 10 further includes a fixing housing 330. The one or more first shim members 310 are the first shim magnet, and the fixing housing 330 is coupled to a surface of the gantry 210. The fixed housing 330 is equipped with a containing cavity 331, and the one or more fixing members 320 and the first shim magnet are disposed within the containing cavity 331. In some embodiments, the one or more fixing members 320 are inserted into the containing cavity 331.

Exemplarily, the one or more fixing members 320 and the fixing housing 330 are connected in manners such as integrally molding, removably connection, or bonding. The fixing housing 330 is fixed to the surface of the gantry 210 in manners such as bonding, removable connected, or integrally molded, which in turn enables the one or more first shim members 310 to be fixed to the surface of the gantry.

In some embodiments, the therapy assembly includes a linear accelerator, a collimator, etc.

The linear accelerator uses a microwave technology to accelerate electrons. In some embodiments, the linear accelerator is operably connected to a microwave device (not shown in the figures). The microwave device is configured to accelerate the electrons in the linear accelerator. In some embodiments, the linear accelerator is operably connected to the microwave device by rotating waveguide. The rotating waveguide keeps the microwave device stationary relative to an MRI device as the linear accelerator rotates about a central axis of the gantry 210 during radiation therapy of an object. In some embodiments, during radiation therapy of the object, the microwave device also rotates along the linear accelerator around the central axis of the gantry 210.

In some embodiments, the radiation therapy system 10 further includes a beam control device and a target. The beam control device is configured to control a radiation particle beam generated by the linear accelerator. In some embodiments, the beam control device includes the collimator.

In some embodiments, the radiation particle beam generated by the linear accelerator is deflected, defocused, or focused, etc., by the beam control device. In some embodiments, the beam control device controls the radiation particle beam to achieve a required position, direction, spatial distribution, energy distribution, beam shape, etc. As used in the present disclosure, the position refers to a point or a region on the target where the electrons in the radiation particle beam collide. The direction refers to a direction in which the electrons of the radiation particle beam are emitted. The spatial distribution refers to a distribution of the electrons in the radiating particle beam in a 3D space. The energy distribution refers to a distribution of the electron energy in the radiation particle beam. The beam shape refers to a cross-sectional shape of the radiation particle beam.

When the accelerated electron beam collides on the target, the target generates a radiation beam for the radiation therapy of the target region of the object. For example, the electron beam emitted from the linear accelerator is deflected onto the target and generates X-rays at a high energy level. In some embodiments, the target is made of a material including aluminum, copper, silver, tungsten, etc., or an alloy thereof, or any combination thereof.

In some embodiments, the radiation therapy system 10 further includes at least one deflector magnet disposed within a first cavity. The deflector magnet is configured to deflect the electrons toward a preset target. When the electrons collide with the preset target, the radiation beam is generated. By using the deflecting magnet to change a trajectory of the electron beam, the electron beam is deflected toward the target along a preset trajectory. The deflecting magnet is a permanent magnet, an electromagnet, etc.

In some embodiments, the radiation therapy system 10 also includes an X-ray imaging flat panel detector, which combines the existing linear accelerator to form an X-ray imaging system, which is used for a tumor positioning etc., to further improve a system performance. When imaging, a ray energy of the linear accelerator is simply adjusted to a kilovolt level in order to meet a contrast required for the imaging.

In one embodiment, the radiation therapy system 10 further includes a radiation shielding assembly surrounding the linear accelerator. The radiation shielding assembly protects the object and at least one component of the magnetic resonance imaging device from the radiation beam generated by the linear accelerator. In some embodiments, the radiation shielding assembly is an annular structure surrounding the linear accelerator. The radiation shielding assembly has an opening for the electron beam emitted by the linear accelerator to pass through. A length of the radiation shielding assembly in a first axial direction is no less than a length of the linear accelerator.

The radiation shielding assembly is made of a material capable of absorbing the radiation beam from the linear accelerator in order to provide a radiation shielding for the object and for the components of the MRI device. Exemplary materials for absorbing the radiation include materials for absorbing photon rays, materials for absorbing particle rays (e.g., neutron rays), etc. The materials for absorbing photon rays include steel, aluminum, lead, tungsten, etc., or alloys thereof, or combinations thereof. The materials for absorbing the neutron rays include boron, graphite, etc., or alloys thereof, or combinations thereof.

In some embodiments, the MRI device generates image data related to magnetic resonance signals generated by scanning the object or a portion of the object. The object includes a body, a substance, a thing, etc., or any combination thereof. In some embodiments, the object includes a particular portion of the body, a particular organ, or a particular tissue, etc. For example, the object includes a head, a brain, a neck, a body, shoulders, arms, a chest, a heart, a stomach, blood vessels, soft tissues, knees, feet, etc. of a patient. According to a type of a magnet of the MRI device, the MRI device includes a permanent magnet MRI scanner, a superconducting electromagnet MRI scanner, or a resistive electromagnet MRI scanner, etc.

In some embodiments, according to a strength of the magnetic field generated by the MRI device, the MRI device includes a high-field MRI scanner, a mid-field MRI scanner, a low-field MRI scanner, etc. In some embodiments, the MRI device is of a closed hole cylindrical type, an open hole cylindrical type, etc.

In some embodiments, the radiation therapy system is a multimodality imaging system including, for example, a magnetic resonance imaging-radiation therapy (MRI-RT) system, a positron emission tomography-radiation therapy (PET-RT) system, etc.

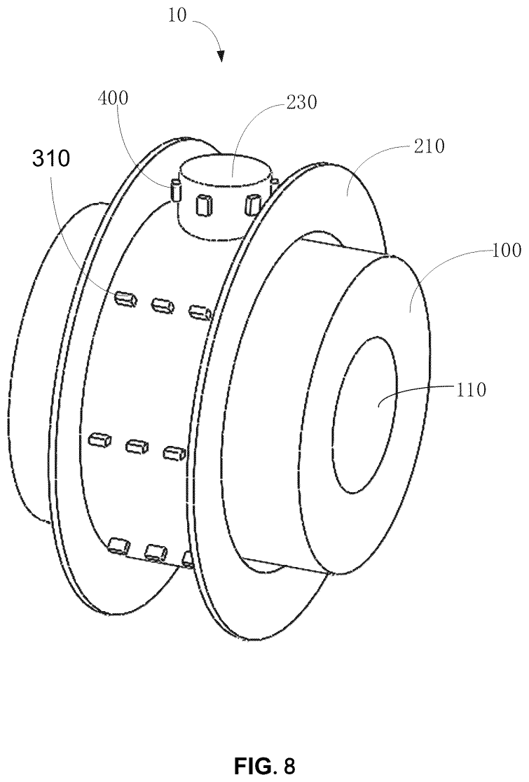

FIG. 8 is a schematic diagram illustrating a 3D structure of an exemplary radiation therapy system according to other embodiments of the present disclosure.

In some embodiments, one or more second shim members 400 are configured to generate a second compensating magnetic field. At a second target position, the second compensating magnetic field is in an opposite direction of the interfering magnetic field, so as to counteract the interfering magnetic field to avoid the interfering magnetic field from affecting the main magnetic field.

In some embodiments, as shown in FIG. 8, the one or more second shim members 400 are arranged around a circumference of the therapy head 230.

As the therapy head 230 is mounted on the gantry 210, and the therapy head 230 has a magnetic component and rotates with the gantry 210. By arranging the one or more second shim members 400 on an outer circumferential wall of the therapy head 230, the one or more second shim members 400 are made to generate the second compensating magnetic field, which counteracts the interfering magnetic field generated by a magnet within the therapy head 230, thereby reducing an effect of the therapy head 230 on the main magnetic field within an imaging region. Meanwhile, as the one or more second shim members 400 are disposed on the circumferential wall of the therapy head 230, there is no change in a relative position between the one or more second shim members 400 and the therapy head 230, i.e., the counteracting effect of the magnetic fields of the one or more second shim members 400 and the therapy head 230 always exists, thereby reducing the interference of the therapy head 230 with the main magnetic field, improving a magnetic field uniformity in the imaging region, and ensuring a desired imaging effect. The one or more second shim members 400 include permanent magnets, electromagnetic coils, or ferromagnetic materials, etc.

In some embodiments, as shown in FIG. 8, the one or more second shim members 400 are arranged along a circumferential direction of the therapy assembly so that the compensating magnetic field generated therefrom is more uniformly distributed along the circumferential direction.

In some embodiments, the one or more second shim members 400 include second shim magnets. There may be a plurality of second shim magnets. The plurality of second shim magnets are distributed in an array along the circumferential direction of the therapy assembly, with distance between two adjacent second shim magnets being equal, i.e., the plurality of the second shim field magnets are evenly arranged along the circumferential direction of the therapy assembly. By arranging the plurality of second shim magnets along the circumferential direction of the therapy assembly, the compensating magnetic field generated therefrom is more uniformly distributed along the circumferential direction, thereby compensating the magnetic field of the therapy assembly to reduce the interference of the magnets within the therapy assembly on the main magnetic field in the imaging region, so that the uniformly of the main magnetic field is better.

It is appreciated that along the circumferential direction of the therapy assembly, the distances between two adjacent second shim magnets may be unequal, i.e., the plurality of second shim magnets are not arranged evenly in the circumferential direction of the therapy assembly. The specific arrangement is adopted based on requirements of actual uses, and an optimal arrangement is obtained through a simulation of the interfering magnetic field using an optimization algorithm.

In some embodiments, different second shim magnets have different geometric characteristics. Exemplarily, a position of a particle beam emitted close to the therapy head 230 is disposed with the second shim magnet with a greater geometric shape, e.g., the second shim magnet has a greater thickness along a radial direction of the gantry 210.

In some embodiments, shim piece(s) are also disposed within the imaging region 110. The shim piece(s) are used to counteract the main magnetic field generated by the superconducting magnet. A cross-section of each of the shim piece(s) has a regular shape such as a square or a circle, which facilitates processing and manufacturing of the third shim member.

It is appreciated that the shim piece(s) may not rotate with the gantry 210. By disposing the shim piece(s) to achieve the requirement for an axial symmetry uniformity correction within a region traversed by a radiation beam of a linear accelerator, the main magnetic field generated by the superconducting magnets is corrected, allowing for an improved magnetic field uniformity in the imaging region, thereby ensuring the imaging effect.

It is noted that the aforementioned first shim member(s), the second shim member(s), and the shim piece(s) are disposed simultaneously in the radiation therapy system, so that a uniform compensating magnetic field is generated, thereby compensating and correcting the main magnetic field generated by the superconducting magnet and the magnetic field generated by the therapy assembly, resulting in a better uniformity of the main magnetic field. The first shim member(s), the second shim member(s), and the shim piece(s) are mounted on a corresponding component using the aforementioned mounting structure, and the cross-sections of the first shim member(s), the second shim member(s), and the shim piece(s) have regular shapes such as squares or circles, which are convenient for the processing and manufacturing.

In some embodiments of the present disclosure, a shimming method of the radiation therapy system is further provided. The shimming method is performed by the radiation therapy system, and the shimming method includes: obtaining a first main magnetic field within the imaging region, the first main magnetic field including a plurality of first magnetic field sub-regions; and for each of the plurality of first magnetic field sub-regions, in response to a difference between a magnetic field strength of the first magnetic field sub-region and a magnetic field strength of another first magnetic field sub-region adjacent to the first magnetic field sub-region being greater than a first preset threshold, determining one or more target shim members in the radiation therapy system. The first preset threshold is preset empirically.

The first main magnetic field is a magnetic field formed by the superconducting magnets before disposing the one or more target shim members. The first main magnetic field is mainly formed by the main magnetic field and is affected by the magnetic components.

The first magnetic field sub-regions are a plurality of sub-regions obtained by dividing the first main magnetic field and are configured for evaluating the magnetic field uniformity of the first main magnetic field.

In some embodiments, obtaining the first main magnetic field within the imaging region includes: establishing a 3D model of the radiation therapy system, and gridding the 3D model and obtaining a finite element model. Simulation software such as ansys is used to perform simulation. After an iterative calculation is completed, a magnetic field layer distribution of each region is generated and displayed on a display device. A magnetic field layer refers to a magnetic field distribution map expressed in different colors.

In some embodiments, if a difference in magnetic field strengths between one first magnetic field sub-region and a magnetic field sub-region adjacent to the first magnetic field sub-region is higher than a first preset threshold, and the magnetic field strength of the first magnetic field sub-region is greater than the magnetic field strength of the adjacent magnetic field sub-region, then the first shim member is placed in the first magnetic field sub-region.

Exemplarily, when the magnet within the linear accelerator has a greater effect on the main magnetic field, i.e., when the difference in the magnetic strengths between the first magnetic field sub-region and the adjacent magnetic field sub-region is relatively large, the color of the layer of that sub-region is different from the color of the layer of the other sub-regions. For example, for the sub-region whose magnetic strength has a greater difference than the adjacent sub-region, the color of the layer is red, while the layers of the other sub-regions are blue, then the first shim member is placed at a position corresponding to the red region, and the 3D modeling and simulation is re-performed.

In some embodiments, the shimming method further includes: determining, based on the first main magnetic field, one or more characteristics of the one or more target shim members using a parameter model, the parameter model being a machine learning model.

In some embodiments, the parameter model is a model for determining the characteristics of the one or more target shim members. For example, the parameter mode is a deep neural network (DNN), etc.

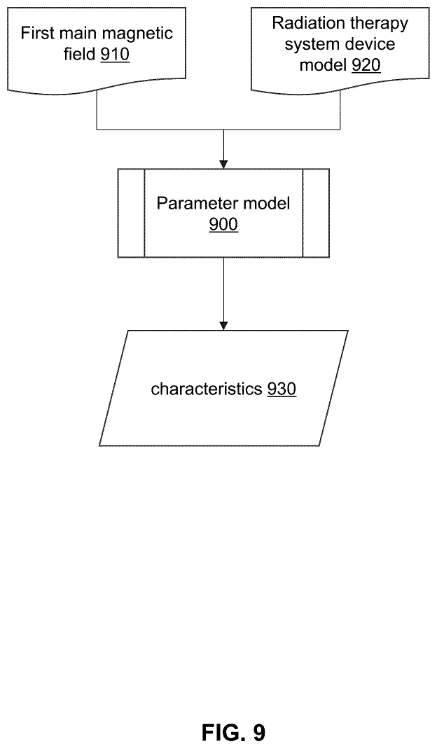

FIG. 9 is a schematic diagram illustrating an application manner of a parameter model according to some embodiments of the present disclosure.

As shown in FIG. 9, inputs to a parameter model 900 include a first main magnetic field 910 and a radiation therapy system device model 920, and outputs of the parameter model include characteristics 930 of one or more target shim members. The first main magnetic field is represented as an image, which is obtained according to an actual measured result. The radiation therapy system device model is a factory parameter, and the radiation therapy system is obtained by reading the factory parameter of a built-in system. The characteristics include the parameter characteristics and the spatial characteristics. More contents on the characteristics are found in above descriptions. The characteristics further include a specification, a placement position, a placement angle, a count, a volume, a mass, an induction strength of magnetic materials, etc. of the one or more target shim members.

In some embodiments, the parameter model is trained by a great count of first training samples with first labels. The first training samples include sample first main magnetic fields and sample radiation therapy system device models, and the first training samples and the first labels are obtained based on historical data. The first labels are actual characteristics of the one or more target shim members corresponding to the sample first main magnetic fields and the sample radiation therapy system device models.

In some embodiments, for the systems of each device model, a shimmed system is set and then one or more target shim members are randomly selected to be removed from the system, so that training data is obtained in order to increase the amount of training data.

Appropriate characteristics of the one or more target shim members obtained according to model prediction have a relatively high accuracy and high efficiency.

In some embodiments, the shimming method further includes: obtaining a second main magnetic field generated after disposing the one or more target shim members, the second main magnetic field including a plurality of second magnetic field sub-regions; for each of the plurality of second magnetic field sub-regions, in response to a difference between a magnetic field strength of the second magnetic field sub-region and a magnetic field strength of another second magnetic field sub-region adjacent to the second magnetic field sub-region being greater than a second preset threshold, adjusting the one or more characteristics of the one or more target shim members. The first preset threshold is preset empirically.

The second main magnetic field is a magnetic field formed by the superconducting magnet after setting up the one or more target shim members. The second main magnetic field is mainly formed by the main magnetic field, and effects of the magnetic components are counteracted by the one or more target shim members.

The second magnetic field sub-regions are a plurality of sub-regions obtained by dividing the second main magnetic field, which are used for evaluating the magnetic field uniformity of the second main magnetic field. Manners of division between the first magnetic field sub-regions and the second magnetic field sub-regions may be consistent or inconsistent.

Further exemplarily, after placing the one or more first shim members in the aforementioned red sub-region, the 3D modeling and simulation is re-performed, and it is determined whether a corresponding layer color is consistent with the layer colors of the other sub-regions. If the corresponding layer color is inconsistent with the layer color of the other sub-regions, the characteristics of the one or more first shim members are continuously adjusted until the difference between the magnetic field strength of the red sub-region and the magnetic field strength of the other sub-regions is smaller.

By verifying the effect of the setting of the one or more target shim members, as well as further adjustments, the shimming effect is better ensured, and the imaging effect is guaranteed.

By arranging the one or more target shim members at suitable positions, the compensating magnetic field generated by the one or more target shim members is made to be able to reduce or eliminate the effect of the interfering magnetic field on the main magnetic field, so that the magnetic field evenness in the imaging region is improved, and thus the imaging effect is ensured.

The basic concepts have been described above, and it is apparent to those skilled in the art that the foregoing detailed disclosure serves only as an example and does not constitute a limitation of the present disclosure. While not expressly stated herein, those skilled in the art may make various modifications, improvements, and amendments to the present disclosure. Those types of modifications, improvements, and amendments are suggested in the present disclosure, so those types of modifications, improvements, and amendments remain within the spirit and scope of the exemplary embodiments of the present disclosure.

Also, the present disclosure uses specific words to describe embodiments of the present disclosure. Such as “an embodiment,” “one embodiment,” and/or “some embodiments” means a feature, structure, or characteristic associated with at least one embodiment of the present disclosure. Accordingly, it should be emphasized and noted that two or more references in the present disclosure, at different positions, to “one embodiment,” or “an embodiment,” or “an alternative embodiment” in different positions do not necessarily refer to the same embodiment. In addition, certain features, structures, or characteristics of one or more embodiments of the present disclosure are suitably combined.

Similarly, it should be noted that in order to simplify the presentation of the disclosure of the present disclosure, and thereby aiding in the understanding of one or more embodiments of the present disclosure, the foregoing descriptions of embodiments of the present disclosure sometimes combine a variety of features into a single embodiment, accompanying drawings, or descriptions thereof. However, this method of disclosure does not imply that the objects of the present disclosure require more features than those mentioned in the claims. Rather, the claimed subject matter may lie in less than all features of a single foregoing disclosed embodiment.

Finally, it should be understood that the embodiments described in the present disclosure are only used to illustrate the principles of the embodiments of the present disclosure. Other deformations may also fall within the scope of the present disclosure. As such, alternative configurations of embodiments of the present disclosure may be viewed as consistent with the teachings of the present disclosure as an example, not as a limitation. Correspondingly, the embodiments of the present disclosure are not limited to the embodiments expressly presented and described herein.

What is claimed is:

1. A radiation therapy system, comprising:
a magnet container configured to form an imaging region;
a therapy assembly including one or more magnetic components, the one or more magnetic components inducing an interfering magnetic field; and
a plurality of target shim members, wherein:
the plurality of target shim members are disposed on the radiation therapy system, each of the plurality of target shim members has parameter characteristics and spatial characteristics, and at least one of the parameter characteristics and the spatial characteristics of at least two of the plurality of target shim members is different; and
the plurality of target shim members are configured to generate a compensating magnetic field, the compensating magnetic field being configured to at least partially counteract the interfering magnetic field.

2. The radiation therapy system of claim 1, wherein the parameter characteristics include a geometric characteristic of each of the plurality of target shim members, the spatial characteristics include position information, the positional information includes a distance between each adjacent two of the plurality of target shim members.

3. The radiation therapy system of claim 1, wherein the therapy assembly includes a gantry disposed around the imaging region, the plurality of target shim members include one or more first shim members, the one or more first shim members are distributed on the gantry, and the one or more first shim members are configured to counteract the interfering magnetic field at a first target position.

4. The radiation therapy system of claim 3, wherein the plurality of target shim members are unevenly distributed on the gantry.

5. The radiation therapy system of claim 4, wherein a distribution density formed by a distribution of the one or more first shim members disposed close to the magnetic components is greater than a distribution density formed by a distribution of the one or more first shim members disposed away from the magnetic components.

6. The radiation therapy system of claim 3, wherein the one or more first shim members are distributed on the outer and/or inner side of the gantry.

7. The radiation therapy system of claim 3, further comprising a shim gantry, the shim gantry being equipped with the plurality of target shim members, the shim gantry being configured to rotate synchronously with the gantry.

8. The radiation therapy system of claim 3, wherein:
the one or more first shim members are configured to generate a first compensating magnetic field; and
at the first target position, the first compensating magnetic field is in an opposite direction of the interfering magnetic field, the first target position being located in the imaging region.

9. The radiation therapy system of claim 3 further comprising one or more first fixing members including at least one first connection element and at least one first fixing support, wherein the at least one first connection element is configured to connect the gantry and the at least one first fixing support, and the at least one first fixing support is configured to fix the one or more first shim members.

10. The radiation therapy system of claim 1, wherein the plurality of target shim members include one or more second shim members distributed on the therapy assembly, and the one or more second shim members are configured to counteract the interfering magnetic field at a second target position, the second target position being located at the therapy assembly or in a region surrounding the therapy assembly.

11. The radiation therapy system of claim 10 further comprising one or more second fixing members, the one or more second fixing members including at least one second connection element and at least one second fixing support, wherein the at least one second connection element is configured to connect the therapy assembly and the at least one second fixing support, and the at least one second fixing support is configured to fix the one or more second shim members.

12. The radiation therapy system of claim 3, wherein:
at least one of the one or more first shim members includes a first shim coil; and
at least one of the one or more first fixing members is connected with the first shim coil.

13. The radiation therapy system of claim 3, wherein:
at least one of the one or more first shim members includes a first shim coil;
when the gantry is rotating, a current passes through the first shim coil; and
when the gantry is at rest, no current passes through the first shim coil.

14. The radiation therapy system of claim 13, wherein:
at least one of the one or more first shim members includes a first shim magnet, and the radiation therapy system further includes a shim shield member; and
the shim shield member is configured to shield a magnetic field generated by the first shim magnet when the gantry is at rest, and un-shield the magnetic field generated by the first shim magnet when the gantry is rotating.

15. The radiation therapy system of claim 3 further comprising a fixing housing, wherein:
at least one of the one or more first shim members includes a first shim magnet, and the fixing housing is connected to the surface of the gantry; and
the fixing housing includes a containing cavity, the one or more first fixing members and the first shim magnet are both disposed in the containing cavity.

16. The radiation therapy system of claim 10, wherein the therapy assembly includes a therapy head, the one or more second shim members are arranged surrounding a circumferential direction along the therapy head.

17. The radiation therapy system of claim 16, wherein:
each of the one or more second shim members includes a second shim magnet;
the one or more second shim magnets are distributed in a circumferential array along the therapy assembly; and
distances between two adjacent second shim magnets are equal, and different the second shim magnets have different geometric characteristics.

18. A shimming method of a radiation therapy system, comprising:
obtaining a first main magnetic field within an imaging region, the first main magnetic field including a plurality of first magnetic field sub-regions; and
for each of the plurality of first magnetic field sub-regions, in response to a difference between a magnetic field strength of the first magnetic field sub-region and a magnetic field strength of another first magnetic field sub-region adjacent to the first magnetic field sub-region being greater than a first preset threshold, determining a plurality of target shim members in the radiation therapy system.

19. The method of claim 18, further comprising:
determining, based on the first main magnetic field, spatial distribution characteristics of the plurality of target shim members using a parameter model, the parameter model being a machine learning model.

20. The method of claim 18, further comprising:
obtaining a second main magnetic field generated after disposing the plurality of target shim members, the second main magnetic field including a plurality of second magnetic field sub-regions; and
for each of the plurality of second magnetic field sub-regions, in response to a difference between a magnetic field strength of the second magnetic field sub-region and a magnetic field strength of another second magnetic field sub-region adjacent to the second magnetic field sub-region being greater than a second preset threshold, adjusting the one or more design parameters of the plurality of target shim members.
